(19) United States
(12) Patent Application Publication
(10) Pub. No.: US 2016/0184201 A1
(43) Pub. Date: Jun. 30, 2016

(54) ANTIPERSPIRANT/DEODORANT COMPOSITIONS

(71) Applicant: COLGATE-PALMOLIVE COMPANY, New York, NY (US)

(72) Inventors: Aixing Fan, Bridgewater, NJ (US); Sandra Wadeer, Flanders, NJ (US); Adam Schatteman, Lake Hopatcong, NJ (US)

(73) Assignee: Colgate-Palmolive Company, New York, NY (US)

(21) Appl. No.: 14/909,053

(22) PCT Filed: Jul. 29, 2013
(86) PCT No.: PCT/US13/52571

§ 371 Date: Jan. 29, 2016

(51) Int. Cl. A61K 8/365; A61Q 15/00

(52) U.S. Cl. CPC A61K 8/365; A61Q 15/00; A61K 2800/10

(57) ABSTRACT

Antiperspirant and/or deodorant compositions containing lactic acid oligomers. The lactic acid oligomers provide a long lasting protection.

### BACKGROUND

Many deodorant compositions use an antimicrobial active to kill microorganisms in axillary (underarm) region to reduce or eliminate body odor caused by microbial, e.g., bacterial, growth in this region. Deodorants can be provided in many forms, such as a roll on, a gel, spray, aerosol, soft solid or as a solid stick. A drawback of some deodorants is loss of active potency over time and regrowth of bacteria that leads to malodor.

It would be desirable to provide a deodorant composition that provides sustained release of the active, i.e., protection over an extended period of time or stronger protection at a later time point.

### BRIEF SUMMARY

A topical anhydrous composition comprising an antibacterial effective amount at least one lactic acid oligomer and a carrier suitable for application to skin.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Lactic Acid Oligomer

The lactic acid oligomers useful in the compositions of the invention typically have a degree of polymerization between 1.8 and 6, more particularly between 2 and 4, and most particularly about 2. The lactic acid oligomer as described herein encompasses both straight-chain and cyclic lactic acid oligomers. In one embodiment, the lactic acid oligomer is lactide, which is cyclic lactic acid dimer. The lactide can be in the D, L, or Meso optical forms. Suitable lactic acid oligomers are commercially available from Purac Biochem BV, Gorinchem, NL.

The lactic acid oligomer as described herein is water-insoluble and is biodegradable. The oligomer is a polyester which reacts with water upon contact therewith, i.e. the water hydrolyses the ester bond. The use of such an oligomer with a degree of polymerization in this range provides a controlled release of water-soluble lactic acid over time when contacted with water or aqueous solutions such as sweat. In this way, the antimicrobial component(s) can properly exert its deodorant function in the initial stage of topical application to the skin as well as in later stages, since use of the lactic acid oligomer ensures that lactic acid is slowly released such that it exerts its function predominantly when sweat is actively produced. This results in longer lasting deodorant protection as compared to a similar composition having lactic acid instead of the oligomer. Further, the oligomer acts as a store or reservoir of lactic acid wherein the release of the lactic acid is triggered by sweat—a smart mechanism where stronger protection is provided when needed more. In addition, by use the oligomer form, free lactic acid in the composition is reduced or eliminated which results in improved formulaic stability, as the acid is not available to react with other composition ingredients.

The lactic acid oligomer is present in an effective antimicrobial amount, that is, an amount the kills or inhibits growth of microorganisms, in particular, bacteria, to a greater extent than a control composition not containing the lactic acid oligomer, when applied to the axillary region as indicated by standard microbiological assays. Such an amount in the deodorant composition is typically about 0.1 to 10%, more particularly 0.5 to 8%, more particularly 1 to 6%, and even more particularly 1 to 3%.

### Antiperspirant Active

When the composition includes an antiperspirant active, any of the known antiperspirant active materials can be utilized in the composition. Antiperspirant actives include, but are not limited to, aluminum chlorhydrate, aluminum chloride, aluminum sesquichlorohydrate, aluminum-zirconium hydroxychlorides, complexes or adducts of the above-mentioned active ingredients with glycol, such as propylene glycol (for example, “Rehydrol” II from Reheis Chemical Co.), and combinations thereof. Known aluminum-zirconium salts in combination with neutral amino acids, such as glycine (e.g., aluminum-zirconium tetrachlorohydrex Gly) can also be used. Generally, any of the Category I active antiperspirant ingredients, listed in the Food and Drug Administration's Monograph on Antiperspirant Drug Products for overall-the-counter human use (Oct. 10, 1973) can be used.

In other embodiments, the antiperspirant active is an aluminum salt and/or an aluminum-zirconium salt, such as those described above, that are further stabilized by betaine and a calcium salt. More information about betaine and calcium salt stabilized antiperspirant salts can be found in U.S. Patent Application Publication No. 2006/0204463 to Tang et al., which is incorporated herein by reference only for the disclosure of the antiperspirant actives.

In other embodiments, the antiperspirant active, such as those described above, is selected to have a low metal to chloride ratio. Examples of these antiperspirant actives can be found in U.S. Pat. No. 6,375,937 to Chopra et al, and in U.S. Pat. No. 7,311,898 to Tang et al., which are incorporated herein by reference only for their disclosure of the antiperspirant active.

In other embodiments, the type of salt of interest, an aluminum zirconium tetrasalt or octasalt free of glycine are used wherein aluminum zirconium salt is stabilized by Betaine and has a metal to chloride ratio of about 0.9:1 to about 1.3:1 (and in other embodiments of about 0.9:1 to about 1.2:1 or about 0.9:1 to about 1.1:1). For the tetrasalt, the Al/Zr atomic ratio can be about 3.2:1 to about 4.1:1.0 and the Betaine:zirconium mole ratio can be about 0.2:1 to about 3.0:1 (or in other embodiments of about 0.4:1 to about 1.5:1). Another salt that can be used is an aluminum chloride salt buffered by Betaine, wherein the salt has a metal to chloride ratio of 0.9:1 to 1.3:1 (and in other embodiments of about 0.9:1 to about 1.2:1 or about 0.9:1 to about 1.1:1). For the octasalt the Al/Zr atomic ratio is about 6.2:1 to about 10.0:1 and the Betaine:Zr mole ratio is about 0.2:1 to about 3.0:1 (or in other embodiments of about 0.4:1 to about 1.5:1). In one embodiment, in the case of a salt that contains zirconium, the Betaine is incorporated during the synthesis of the salt so as to maximize the stabilizing effect this ingredient has (especially on the zirconium species). Alternatively, it can be post added to a glycine-free salt along with additional active phase ingredients to form a Betaine stabilized active.

Examples of commercially available glycine-free low M:Cl ratio tetrasalts and octasalts include, but are not limited to, REZAL™ AZP 955 CPG and REZAL™ AZP 885 respectively (both from Reheis Chemical Company, Berkeley Heights. N.J.). A more detailed description of making such commercially available salts can be found for example, in U.S. Pat. Nos. 7,074,394 and 6,960,338. Further examples of making these types of salt complexes are described in U.S. Patent Application Publication No. 2004/0198998 and U.S. Pat. No. 7,105,691.

In addition to the anti-irritation properties of Betaine, it has also been found that antiperspirant formulations preserve their fragrance stability upon ageing when the Al/Zr salt is used in association with Betaine.

Additionally, the antiperspirant active can be a calcium salt stabilized antiperspirant active. Examples of calcium salt stabilized antiperspirant actives can be found in U.S. Pat. No. 7,704,531, which is incorporated herein by reference only for the disclosure of the calcium salt stabilized antiperspirant actives.

In addition, any new ingredient, not listed in the Monograph, such as aluminum nitratohydrate and its combination with zirconyl hydroxychlorides and nitrates, or aluminum-stannous chlorohydrates, can be incorporated as an antiperspirant active. Antiperspirant actives can include, but are not limited to, the following: astringent salt of aluminum, astringent salt of zirconium, aluminum bromohydrate, aluminum chlorohydrate, aluminum dichlorohydrate, aluminum sesquichlorohydrate, aluminum chlorohydrex PG, aluminum dichlorohydrex PG, aluminum sesquichlorohydrex PG, aluminum chlorohydrex PEG, aluminum dichlorohydrex PEG, aluminum sesquichlorohydrex PEG, aluminum chloride, aluminum sulfate, aluminum zirconium chlorohydrate, aluminum zirconium trichlorohydrate, aluminum zirconium tetrachlorohydrate, aluminum zirconium pentachlorohydrate, aluminum zirconium octachlorohydrate, aluminum zirconium tetrachlorohydrex propylene glycol, aluminum zirconium trichlorohydrex Gly, aluminum zirconium tetrachlorohydrex Gly, aluminum zirconium pentachlorohydrex Gly, aluminum zirconium octachlorohydrex Gly, buffered aluminum sulfate, potassium alum, sodium aluminum chlorohydroxy lactate. In one embodiment, the antiperspirant active is aluminum chlorhydrate. In another embodiment, the antiperspirant active is aluminum zirconium tetrachlorohydrex propylene glycol.

In one embodiment, the amount of antiperspirant active is about 0.1 to 30% of the composition, in another embodiment 7 to 25%, in another embodiment 9 to 25%.

### Surfactants

Any surfactant that can be used in antiperspirant and/or deodorant compositions can be included. Examples of the surfactant include, but are not limited to, nonionic surfactants, silicone surfactants, and combinations thereof.

Nonionic surfactants that can be used include, but are not limited to, (a) sorbitan esters and ethoxylated sorbitan esters (for example PEG-20 sorbitan isostearate, sorbitan monolaurate, polysorbate-20, polysorbate-40, polysorbate-60, polysorbate-80); (b) ethoxylates (for example, Ceteth-20, PEG-30 castor oil, PEG-40 hydrogenated castor oil, PEG-60 hydrogenated castor oil, Laureth-7, Isolaureth-6, Steareth-10, Steareth-20, Steareth-21, Steareth-100, Ceteareth-12, Oleth-5, Oleth-10); (c) ethoxylated adducts (for example, PEG-25 stearate, glyceryl stearate and PEG-100 stearate); (d) PEG esters (for example, PEG-8 oleate, PEG-8 laurate, PEG-8 dilaurate, PEG-12 dilaurate, PEG-80 diisostearate, PEG-40 stearate): (e) propoxylates (for example. PPG-10 butanediol, PPG-50 oleyl ether, PPG-2-ceteareth-9, PPG-3-deceth-3, PPG-5-ceteth-20); (f) ethoxylated modified triglycerides (for example, PEG-20 corn glycerides, PEG-12 palm kernel glycerides); (g) alkylphenol aromatic ethoxylates (for example, dinonylphenol ethoxylate with 9 moles of EO, octylphenol ethoxylate with 20 moles of EO, octylphenol ethoxylate with 40 moles of EO); (h) block copolymers that are alkoxylated glycols having ethoxylated and propoxylated segments (for example, POLOXAMER™ 182 and 234, POLOXAMER™ 105 Benzoate, and MEROXAPOL™ 174); and combinations thereof. In one embodiment, the nonionic surfactant is selected so that it has an HLB (hydrophilic-lipophilic balance) value of 8-16 (more particularly 8-12).

In one embodiment, the nonionic surfactant is selected from ethoxylated non-ionic surfactants and propoxylated non-ionic surfactants. Examples of these include, but are not limited to Steareth 2, Steareth 20, and Steareth 21. In an oil in water composition embodiment, a combination of 2 surfactants, one having an HLB value of about 2 to about 8 (such as Steareth 2) and the other having an HLB of about 9 to about 18 (such as Steareth 20 and 21), can be used.

Examples of silicone surfactants can be found in U.S. Pat. No. 6,485,716, which is incorporated herein by reference only for the listing of the silicone surfactants. Suitable silicone surfactants include silicone polyglucosides (for example, octyl dimethicone ethoxy glucoside) and silicone copolyols having an HLB value (hydrophilic lipophilic balance)≦8. The HLB value may be measured in a variety of ways such as described in conventional references or found listed in tables of data recording such values. It is intended that any type of HLB measurement technique may be used.

The surfactant can be included in any desired amount. In one embodiment, the amount of surfactant is about 0.1 to 15% of the composition, in another embodiment 2 to 12% of the composition, in another embodiment about 3 to 10%, in another embodiment about 2 to 5%. The amount in the composition is based on the as supplied material.

### Particulates

The composition may also contain particulates which include but are not limited to talc, mica, fragrance encapsulates, or hydrophobically modified starches, such as aluminum starch octenyl succinate (MACKADERM™ ASTRO-DRY™ from McIntyre Group Ltd.). If the composition is in a liquid form and dispensed through a roll-on applicator, the average particle size of the suspended material is sized so that it can pass through the application to prevent the ball applicator from malfunctioning. Usually, the average particle size does not exceed 150 microns.

In one embodiment, the amount of particulates is about 0.1 to 30% of the composition, in another embodiment 1 to 20%, in another embodiment 5 to 15%.

### Malodor Counteracting Agents

In certain embodiments, the composition may also contain as an optional ingredient at least one malodor counteracting alpha, beta-unsaturated ester or mixtures of such materials. In certain embodiments, the level of malodor counteracting composition to deliver a perceivable odor control benefit when delivered from an antiperspirant and/or deodorant composition is about 0.05 to about 0.45 weight % based on the entire composition. The alpha, beta-unsaturated ester malodor counteracting materials are incorporated within the oil phase of an antiperspirant composition. Example of these malodor counteracting components can be found in U.S. Pat. No. 6,610,648 and U.S. Pat. No. 6,495,097, which are incorporated herein only for their disclosure of the alpha, beta unsaturated esters. For example, in this invention the odor neutralizing alpha, beta unsaturated ester mixture demonstrates unexpected stability in antiperspirant compositions containing low metal:chloride (M:Cl) ratio salts free of glycine.

Examples of the alpha, beta unsaturated ester can be found in WO2005/025523, which was filed in the United States as U.S. Application Publication No. 2007/0196308, both of which are incorporated herein by reference to the extent that they do not conflict with the disclosure in this specification.

In one embodiment, the amount of malodor counteracting agent is about 0.05 to 20% of the composition, in another embodiment 0.1 to 20% of the composition, in another embodiment 0.5 to 15%.

The composition of the invention further comprises a carrier suitable for application to the skin. Such carriers include, but are not limited to, volatile silicones, emollients, lipophilic carrier materials or any combination of two or more thereof. The amount of the carrier material can vary widely depending on the type(s) of carrier, therefore the carrier can be present in a quantity of about 0.1 to 98% of the composition.

Volatile silicones

In one embodiment of the composition also comprises at least one volatile silicone component. Volatile compounds in the context of the invention are compounds which volatilize at body temperature, typically having a flash point of 100° C. or less. Suitable volatile silicones, which may be linear, branched or cyclic, are described in Todd et al. “Volatile Silicone Fluids for Cosmetics”, Cosmetics and Toiletries, pp. 27-32 (1976). Silicones containing 3 to 7 and more particularly 4 to 6 silicon atoms are preferred for the purposes of the invention. Particularly preferred are cyclic polydimethylsiloxanes such as, for example, octamethyl cyclotetrasiloxane, decamethyl cyclopentasiloxane or dodecamethyl cyclohexasiloxane which are known as cyclomethicones. They are commercially obtainable from G.E. Silicones as Cyclomethicone D-4 and D-5, from Dow Corning Corp. as Dow Corning® 344, 345 and 244, 245, 246, from General Electric Co. as GE® 7207 and 7158. One embodiment of the linear volatile silicones are those containing 1 to 7 and more particularly 2 to 3 silicon atoms. In another embodiment, the emollient is a volatile silicone is cyclomethicone or trisiloxane.

The volatile silicones when present are typically present in a quantity of about 0.1 to 98% of the composition, more particularly in a quantity of about 1 to 90%, more particularly 5 to 70%, and more particularly in a quantity of 10 to 35%.

### Emollients

The composition can contain non-volatile emollients in any desired amount to achieve a desired emollient effect. In one embodiment, the amount of emollients is up. Emollients are known in the art and are used to impart a soothing effect on the skin. Classes of non-volatile emollients include non-silicone and silicone emollients. Non-volatile, non-silicone emollients include C₁₂-₁₅ alkyl benzoate. Non-volatile silicone material can be a polyethersiloxane, polyalkyarylsiloxane or polyethersiloxane copolymer. An illustrative non-volatile silicone material is phenyl trimethicone. Non-limiting examples of emollients can be found in U.S. Pat. No. 6,007,799. Examples include, but are not limited to, PPG-14 butyl ether, PPG-15 stearyl ether, PPG-3 myristyl ether, stearyl alcohol, stearic acid, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl) succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, alkyl benzoate, hydrocyethyl stearate amide, and hydrogenated polyisobutene.

In one embodiment, In one embodiment, the emollient is selected from linear silicones, cyclic silicones, hydrocarbons, polyhydroxy alcohols having more than 3 carbon atoms, liquid or solid polyalkyleneglycol ethers containing a polypropylene glycol (PPG) moiety and terminating in an alkyl ether, and combinations thereof. In another embodiment, the emollient is a nonvolatile silicone, such as dimethiconol or a longer chain dimethicone.

In one embodiment, the amount of emollient is about 0.1 to 30% of the composition, in another embodiment 1 to 25%, in another embodiment 1 to 15%.

### Lipophilic Carrier Material.

The composition may also contain a lipophilic carrier comprising fat(s), oil(s), wax(s) or a combination thereof. These lipophilic components have structuring properties and provide the composition with the required consistency and with a particularly pleasant skin feel.

Any fats and fat-like substances may be used as part or all of the lipophilic carrier. These include inter alia fats (triglycerides), mono- and diglycerides, fatty alcohols, fatty acids, esters and/or ethers of fatty alcohols and fatty acids and also fatty acid amides or mixtures of these substances.

Waxes are understood to be natural or synthetic materials with the following properties: they are solid or fragile and hard in consistency, coarsely to finely crystalline, transparent or opaque and melt above 30° C. without decomposing. They are low in viscosity and non-stringing only slightly above their melting point and show highly temperature-dependent consistency and solubility. Waxes suitable for use in accordance with the present invention are, for example, natural vegetable waxes with a melting point of 30 to 150° C. such as, for example, candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. Natural waxes usable in accordance with the invention also include the mineral waxes, such as ceresine and ozocerite for example, or the petrochemical waxes, for example petrolatum, paraffin waxes and microwaxes. Other suitable wax components are chemically modified waxes, more particularly the hard waxes such as, for example, montan ester waxes, sasol waxes and hydrogenated jojoba waxes. Synthetic waxes usable in accordance with the invention include, for example, wax-like polyalkylene waxes and polyethylene glycol waxes.

The wax component may also be selected from the group of esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols and also from the group of lactides of long-chain hydroxycarboxylic acids providing the wax component or all the wax components melt at 30 to 150° C. Wax components such as these include, for example, C16-40 alkyl stearates, C20-40 alkyl stearates (for example Kesterwachs® K82H), C20-40 dialkyl esters of dimer acids, C18-38 alkyl hydroxystearoyl stearates or C20-40 alkyl erucates. Other suitable wax components which may be used with advantage are C30-50 alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate. Silicone waxes may also be used.

Suitable oil components are those in which the solids are homogeneously dispersed. A combination of nonpolar and polar oil component is possible. The nonpolar liquid oil components which can make up most of the carrier material include silicone oils and hydrocarbons which may be linear, branched or cyclic. Suitable hydrocarbons are, for example, isohexadecane, isododecane, polydecene and mineral oils such as, for example, thickly liquid and thinly liquid paraffins.

Examples of lipophilic components are described in U.S. Pat. Nos. 7,976,829; 6,849,251, and US patent application no. 2011/0274637, all incorporated herein by reference for their description of lipophilic components, e.g., fats, oils and waxes.

The lipophilic carrier material can be present in a total quantity of about 0.1 to 60% of the composition, in another embodiment 1 to 50%, in another embodiment 1 to 20% and in another embodiment 5 to 15%.

### Other Deodorant Actives

The composition may contain other deodorant actives in addition to the lactic acid oligomer, any known deodorant active can be used. Examples of other deodorant active include, but are not limited to other antimicrobial actives, alcohols, 2,4,4′-trichloro-2′-hydroxy diphenyl ether (Triclosan), octoxyglycerin (SENSIVA™ SC 50), benzethonium chloride, polyhexamethylene biguanides, triethylcitrate, 2-amino-2-methyl-1-propanol (AMP), cetyl-trimethylammomium bromide, cetyl pyridinium chloride, bactericides, and bacteriostats.

In one embodiment, the amount of other deodorant active is about 0.1 to 30% of the composition, in another embodiment 0.1 to 15%, in another embodiment 0.1 to 10%.

### Other Ingredients

A variety of fragrances can be used in these compositions if a scented product is desired. Fragrances can be used in an amount in the range of 0.5% to 2%, particularly 0.01 to 2.0%, and, for example, at a level of about 1%.

The composition of the invention is typically anhydrous, i.e., containing no more than 1% added water (excluding any waters of hydration), more typically no added water. It is anticipated that any waters of hydration in the various ingredients would give a water content of the entire composition of less than 7.5 weight %, e.g. less than 5% or less than 2%.

Conventional gelling agent(s) may be incorporated into the compositions of the invention, for example, stearyl alcohol and dibenzylidene sorbitol. When present, gelling agents are typically present in an amount of about 0.1 to 30%, in another embodiment 7 to 15%. Gel products may also be made using polymers, for example polyethylene glycol.

Some ingredients listed herein can provide more than one function to the compositions. For example, certain emollients can act as lipophilic carrier material and a gelling agent at the same time.

In a first embodiment, the invention provides topical anhydrous composition comprising an effective amount of a deodorant antimicrobial ingredient comprising at least one lactic acid oligomer in combination with a carrier suitable for application to the skin (“Composition 1”), for example

1.1 Composition 1, wherein the degree of polymerization of the lactic acid is between 1.8 and 6.
1.2 Composition 1 wherein the degree of polymerization of the lactic acid is between 2 and 4.
1.3 Composition 1 wherein the degree of polymerization of the lactic acid is 2, i.e., lactide which is 3,6-dimethyl-1,4-dioxane-2,5-dione.
1.4 Any of the foregoing compositions wherein the amount of lactic acid oligomer is about 0.1 to 10%, or 0.5 to 8%, or 1 to 6%, or 1 to 3%, based on the total weight of the composition.
1.5 Any of the foregoing compositions further comprising a carrier suitable for application to the axillary region.
1.6 Any of the foregoing compositions wherein the carrier comprises a volatile silicone, emollient, a lipophilic carrier material or any combination of two or more thereof.
1.7 Any of the foregoing compositions wherein the composition additionally comprises at least one antiperspirant active.
1.8 Composition 1.7 wherein the antiperspirant active is an aluminum zirconium salt.
1.9 Composition 1.7 or 1.8 wherein the amount of antiperspirant active is about 0.1 to 30% or 7 to 25%, or 9 to 25%, based on the total weight of the composition.
1.10 Any of the foregoing compositions wherein the carrier comprises at least one emollient.
1.11 Composition 1.10 wherein the emollient is a C₁₂-₁₅ alkylbenzoate, hydrogenated polyisobutene, myristyl stearate, or a combination thereof.
1.12 Composition 1.10 or 1.11 wherein the amount of emollient is about 1 to 90% or 5 to 70%, or 10 to 35%, based on the total weight of the composition.
1.13 Any of the foregoing compositions wherein the carrier comprises at least one volatile silicone.
1.14 Composition 1.13 wherein the volatile silicone is dimethicone copolyol, cyclomethicone, trisiloxane, or a combination thereof.
1.15 Composition 1.13 or 1.14 wherein the amount of volatile silicone is about 1 to 90%, or 5 to 70% or 10 to 35% based on the total weight of the composition.
1.16 Any of the foregoing compositions wherein the composition additionally comprises at least one particulate in an amount of about 0.1 to 30% or 1 to 20% or 5 to 15%, based on the total weight of the composition.
1.17 Any of the foregoing compositions wherein the composition additionally comprises at least one surfactant in an amount of about 0.1 to 15% or 2 to 12% or 3 to 10%, or 2 to 5%, based on the total weight of the composition.
1.18 Any of the foregoing compositions wherein the composition additionally comprises at least one malodor counteracting agent in addition to the lactic acid oligomer in an amount of about 0.05 to 20% or 0.1 to 20% or 0.5 to 15%, based on the total weight of the composition.
1.19 Any of the foregoing compositions wherein the carrier comprises at least one lipophilic carrier material in an amount of about 0.1 to 60% or 1 to 50% or 1 to 20% or 5 to 15%, based on the total weight of the composition. 1.20 Any of the foregoing compositions wherein the composition additionally comprises at least one fragrance in an amount of about 0.5% to 2% or 0.01 to 2.0% or 1%, based on the total weight of the composition.
1.21 Any of the foregoing compositions wherein the composition additionally comprises at least one gelling agent, in particular in an amount of about 0.1 to 30% or 7 to 15%, based on the total weight of the composition.
1.22 Any of the foregoing compositions in the form of a soft solid or solid stick deodorant product suitable for application to the axillary area.
1.23 Any of the foregoing compositions not containing dibenzyl monosorbitol acetal.
1.24 Any of the foregoing compositions wherein by anhydrous in meant a composition containing no more than 1% added water (excluding any waters of hydration), e.g., no added water, and wherein any waters of hydration in the various ingredients would give a water content of the entire composition of less than 7.5 weight %, e.g., less than 5%, e.g. less than 2%.
1.25 Any of the foregoing Compositions for use as, or in the in the manufacture of, a deodorant.

The compositions of the invention can be prepared by conventional techniques known in the art. For example, for sticks, the lipophilic carrier material and surfactants can be heated together to about 60-90° C. until a clear melt is formed; the lactic acid oligomer and other ingredients can then be incorporated into the lipophilic phase with stirring; the hot melt can then be cooled with stirring to room temperature and then introduced into a stick tube suitable for application to the skin.

Deodorant compositions according to the present invention can be packaged in conventional containers, using conventional techniques. The composition of the invention can be in the form of sprays, aerosols, lotions, roll-ons (in liquid form), gels, creams, soft solids, or solids (e.g., sticks). As the compositions are substantially anhydrous, the compositions are typically solids or soft solids, although anhydrous liquid or gel carriers may be used to make spray, gel, or roll-on products. Where a gel, cream or soft-solid cosmetic composition is produced, the composition can be introduced into a dispensing package (for example, conventional packages for gels with glide on applicators, jars where the gel or cream is applied by hand, and newer style packages having a top surface with pores) as conventionally done in the art. Thereafter, the product can be dispensed from the dispensing package as conventionally done in the art, to deposit the active material, for example, on the skin. For sticks, sprays, aerosols and roll-ons the compositions can be placed in a conventional type of container (with the inclusion of propellants in aerosols). This provides good deposition of the active material on the skin.

Compositions of the present invention can be formulated as clear, translucent or opaque products. A desired feature of the present invention is that a clear, or transparent, cosmetic composition, (for example, a clear or transparent deodorant or antiperspirant composition) can be provided. The term clear or transparent according to the present invention is intended to connote its usual dictionary definition; thus, a clear liquid or gel antiperspirant composition of the present invention allows ready viewing of objects behind it. By contrast, a translucent composition, although allowing light to pass through, causes the light to be scattered so that it will be impossible to see clearly objects behind the translucent composition. An opaque composition does not allow light to pass there through. Within the context of the present invention, a gel or stick is deemed to be transparent or clear if the maximum transmittance of light of any wavelength in the range 400-800 nm through a sample 1 cm thick is at least 35%, or at least 50%. The gel or liquid is deemed translucent if the maximum transmittance of such light through the sample is between 2% and less than about 35%. A gel or liquid is deemed opaque if the maximum transmittance of light is less than about 2%. The transmittance can be measured by placing a sample of the aforementioned thickness into a light beam of a spectrophotometer whose working range includes the visible spectrum, such as a Bausch & Lomb Spectronic 88 Spectrophotometer. As to this definition of clear, see European Patent Application Publication No. 291,334 A2. Thus, according to the present invention, there are differences between transparent (clear), translucent and opaque compositions.

The compositions of this invention may be used to formulate deodorants which are well tolerated by consumers having sensitive skin. Such deodorants include solids such as sticks and creams (creams sometimes being included in the term “soft solid”), gels, liquids (such as are suitable for roll-on products), and aerosols. The forms of these products may be suspensions or emulsions.

The present invention additionally embraces a method of inhibiting or reducing malodor by topically applying an effective amount of a deodorant composition as described herein, e.g., any of Compositions 1, et seq., to the skin of a human user, for example to the axilla, where such reduction in malodor is desired by the user. An effective amount is that amount which reduces malodor to a degree that is noticeable by the user. Typically, the amount of deodorant composition applied will range from about 0.1 gram to about 1.0 gram per axilla depending on the formulation or such amount as will deliver about 0.0001 to about 0.1 gram of deodorant active per axilla. In a particular embodiment, the user is a person having sensitive skin, e.g., a person who has experienced skin irritation or redness following application of a conventional antiperspirant or deodorant.

### EXAMPLES

### Example 1

### Antimicrobial Activity

### Preparation

1. Formulate a 1% or 10000 ppm and 3% or 30000 ppm solution of the neat L-Lactide active tested by weight and vortex until homogeneous in a 50 mL centrifuge tube.
2. Place solutions in shaking incubator at 200 rpm and 37° C. for 1 hour.
3. Repeat the formulation of the solution and shaking incubation for 4 hour and 24 hour time points.
4. Label 96-well plate to indicate positive and negative controls and the active tested against an odor causing bacteria, Staphyloccus haemolyticus.
5. Using a 96-well plate, pipette 100 uL of Tryptic Soy Broth (TSB) into all of the wells.
6. In the first well, add 100 uL of the active solution or desired solvent for positive and negative controls to achieve a total of 200 uL in the first column of wells.
7. Set the multi-channel pipette to 100 uL and begin mixing the contents in the first column of wells and transfer 100 uL of the mixture into each successive well creating two-fold serial dilutions.
8. Repeat until the 12th column is reached making sure to dispose the 100 uL from the last well leaving 100 uL in all of the wells.
9. In the negative control wells add 100 uL of Phosphate Buffered Saline (PBS). Add 100 uL of bacteria (optical density equals to 0.1) to all other wells.
10. Incubate 96-well plate overnight at 37° C. or at room temperature for 48 hours.
11. Minimum inhibition concentration of the sample is determined by measuring the optical density of the 96 plate. Wells of the 96-well plate on an agar plate are streaked to visually confirm the concentrations leading to complete bacteria kill.

### Results

### 1% L-Lactide

For 1% L-Lactide solution at 1 hour of shaking incubation, the first well in the 96-well plate, containing a 4-fold dilution of the sample, exhibited some bacterial growth. After 4 hours of incubation the same well shows complete bacteria kill. Over time the L-Lactide solution proves to be more potent in inhibiting bacteria growth.

### 3% Lactide

The 3% L-Lactide solution after 1 hour of shaking incubation shows no bacterial growth in wells 1 and 2 in the 96-well plate where the dilution factor is 4 and 8 fold respectively. The trend continues after 4 hours of shaking incubation. Finally, after 24 hours of shaking incubation, the bacteria growth is prevented in the third well, where the dilution is 16 fold. The longer the L-Lactide active is incubated, the more potent it becomes and thus the active can prevent bacteria growth over extended periods of time.

1. A topical anhydrous composition comprising an antibacterial effective amount at least one lactic acid oligomer and a carrier suitable for application to skin.

2. The composition of claim 1 wherein the at least one lactic acid oligomer is a 2 to 4 oligomer of lactic acid.

3. The composition of claim 1, wherein the at least one lactic acid oligomer is a lactide.

4. The composition of claim 1, wherein the at least one lactic acid oligomer is L-lactide.

5. The composition of claim 1, further comprising at least one antiperspirant active.

6. The composition of claim 1 further comprising at least one deodorant active in addition to the lactic acid oligomer.

7. The composition of claim 1 wherein the carrier is suitable for application to the axillary region and comprises a volatile silicone, emollient, lipophilic carrier material or an combination of two or more thereof.

8. The composition of claim 1, comprising 0.01 to 5% of the at least one lactic acid oligomer, by weight based on the total weight of the composition.

9. The composition of claim 1, wherein said composition is a solid or soft-solid deodorant product suitable for application to the axillary region.

10. A method for reducing and/or regulating body odor comprising applying an antibacterial effective amount of the composition of claim 1 onto the skin of a user.

11. The method of claim 10 wherein the composition is applied to the axillary area and the user is a person having sensitive skin.
